(19) United States
(12) Patent Application Publication
(10) Pub. No.: US 2016/0115543 A1
(43) Pub. Date: Apr. 28, 2016

(54) Neurosurgical Genomics

(71) Applicant: The Arizona Board of Regents on Behalf of The University of Arizona, Tucson, AZ (US)

(72) Inventors: Martin E. Weinard, Tucson, AZ (US); Matthew J. Gallek, Vail, AZ (US); Jesse Skoch, Tucson, AZ (US)

(21) Appl. No.: 14/894,963

(22) PCT Filed: May 29, 2014
(86) PCT No.: PCT/US14/40045

§ 371 Date: Nov. 30, 2015

Related U.S. Application Data
(60) Provisional application No. 61/828,595, filed on May 29, 2013.
(60) Provisional application No. 61/870,768, filed on Aug. 27, 2013.

(51) Int. Cl. C12Q 1/68

(52) U.S. Cl. CPC C12Q 1/6883; C12Q 2600/106; C12Q 2600/158

(57) ABSTRACT

Ten (10) genes and seven (7) RNA probes are disclosed, the relative expressions of which, hold predictive value with regard to seizure-free surgical outcomes for epilepsy patients. Whole genome and targeted gene expression analyses demonstrate that (a) specific biological process pathways existing in brain tissue of patients with medically intractable temporal lobe epilepsy, and (b) temporal cortical gene expression, differ between patients rendered seizure-free compared to non-seizure-free patients following anterior temporal lobectomy with amygdalohippocampectomy (ATL/AH).

### GOVERNMENT LICENSE RIGHTS

This invention was made with government support under Grant No. R01 MH065151 awarded by the National Institutes of Health (NIH). The government has certain rights in the invention.

### FIELD OF THE INVENTION

In certain embodiments, the invention is directed to use of gene expression to predict seizure outcome following temporal lobectomy.

### BACKGROUND OF THE INVENTION

Epilepsy is one of the most common neurological disorders and affects 2 to 4 million people or approximately 1% of the population of the United States. Treatment of epilepsy may include antiepileptic medications, diet modifications, vagus nerve stimulation, surgical disconnection of epileptic pathways or resective surgery.

Appropriate medications can control seizures in approximately 70% of cases. The remaining 30% of patients, with refractory seizures, may consider surgical intervention for treatment of their epilepsy.

Between 52% and 84% of patients that have surgical intervention have remission of seizures. The most commonly performed operation for treatment of medically intractable seizures is amygdalohippocampectomy with or without resection of additional temporal lobe tissue, such as anterior temporal lobectomy. Approximately 65% of patients with medically intractable temporal lobe epilepsy treated with ATL/AH are rendered seizure-free. Human brain tissue obtained during en bloc temporal lobectomy and amygdalohippocampectomy provides unique opportunities to study fundamental biological processes involved in the pathophysiology of medically intractable temporal lobe epilepsy.

### SUMMARY OF THE INVENTION

Human brain tissue obtained during en bloc temporal lobectomy and amygdalohippocampectomy provides unique opportunities to study fundamental biological processes involved in the pathophysiology of medically intractable temporal lobe epilepsy and to study genomic expression for prognostic value in predicting seizure-free outcome following AH/ATL.

Whole genome and targeted gene expression analyses demonstrate that (a) specific biological process pathways existing in brain tissue of patients with medically intractable temporal lobe epilepsy, and (b) temporal cortical gene expression, differ between patients rendered seizure-free compared to non-seizure-free patients following anterior temporal lobectomy with amygdalohippocampectomy (ATL/AH).

Ten (10) genes and seven (7) RNA probes are identified in this invention the relative expression of which hold predictive value with regard to seizure-free surgical outcome. In addition, this invention identifies six biological process pathways to be different between the two groups.

### BRIEF DESCRIPTION OF THE DRAWINGS

The invention will be better understood from a reading of the following detailed description taken in conjunction with the drawings in which like reference designators are used to designate like elements, and in which:

FIGS. 1-17 illustrate statistical plots of the ten (10) genes and seven (7) single nucleotide polymorphisms (SNPs) described in this invention which have prognostic significance for predicting seizure outcome following ATL/AH;

FIG. 18 graphically illustrates Applicants' method to determine the genetic expression of specific genes in the brain tissue of patients with medically intractable temporal lobe epilepsy, wherein those genetic expressions provide accurate predictors of which patients would be rendered seizure-free following ATL/AH. This Figure illustrates as an example six (6) of the ten (10) genes described in this invention;

FIG. 19 summarizes the statistical significance of the ten (10) genes and seven (7) single nucleotide polymorphisms (SNPs) with prognostic value for seizure outcome following ATL/AH; and

FIG. 20 delineates significant genes and RNA probes (SNPs) identified by logistic regression with prognostic value for seizure outcome following ATL/AH.

### DETAILED DESCRIPTION OF PREFERRED EMBODIMENTS

This invention is described in preferred embodiments in the following description with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of the invention may be combined in any suitable manner in one or more embodiments. In the following description, numerous specific details are recited to provide a thorough understanding of embodiments of the invention. One skilled in the relevant art will recognize, however, that the invention may be practiced without one or more of the specific details, or with other methods, components, materials, and an forth. In other instances, well-known structures, materials, or operations are not shown or described in detail to avoid obscuring aspects of the invention.

Applicants have discovered the first evidence for predictive value of temporal cortical gene expression for seizure outcome after anterior temporal lobectomy and amygdalohippocampectomy (ATL/AH). The invention was developed based on retrospective analysis of prospectively obtained and stored human blood and brain tissue samples harvested through the University of Arizona Human Subjects Protection Program approved protocol.

Gene expression analyses have been performed to elucidate molecular mechanisms involved in the pathophysiology of epilepsy. However, to date there are no studies that investigate the differences in gene expression between surgical patients that are rendered seizure-free and those who continue to have seizures. Applicants predictive method differentiates the gene expression of brain tissue in patients who are rendered seizure-free with the gene expression of those who continue to have seizures following ATL/AH. Applicants' method provides a first instance of “neurosurgical genomics,” wherein gene expression is used as a biomarker with prognostic value predicting successful outcome following operative neurosurgical intervention.

Using prior art methods, temporal lobectomy candidates may be selected with a variety of concordant seizure focus localizing diagnostic studies which may include non-invasive and invasive video/EEG monitoring, MRI brain scan, positron emission tomography (PET) scanning and neuropsychological testing. Gene expression analyses have been performed in the development of this invention which describes molecular mechanisms and genes involved in the pathophysiology of epilepsy and which are associated with seizure outcome following AH/ATL.

To date there are no prior studies that investigate the differences in biological pathways and/or gene expression between surgical patients that continue to have seizures and those that are in remission (i.e. seizure-free outcome). Applicants' method utilizes expression of individual genes and biological process pathways derived from brain tissue in temporal lobectomy subjects to predict which patients will continue to have seizures and those who will have remission of seizures following ATL/AH.

Implementation of the concept of targeted gene expression having predictive value for seizure outcome in temporal lobectomy patients was established in nineteen patients who underwent ATL/AH to treat medically intractable complex partial seizures of temporal lobe origin. From the lateral temporal cortex tissue resected in each patient, isolated total RNA samples were used to produce labeled target for gene expression and specific biological process pathway analyses.

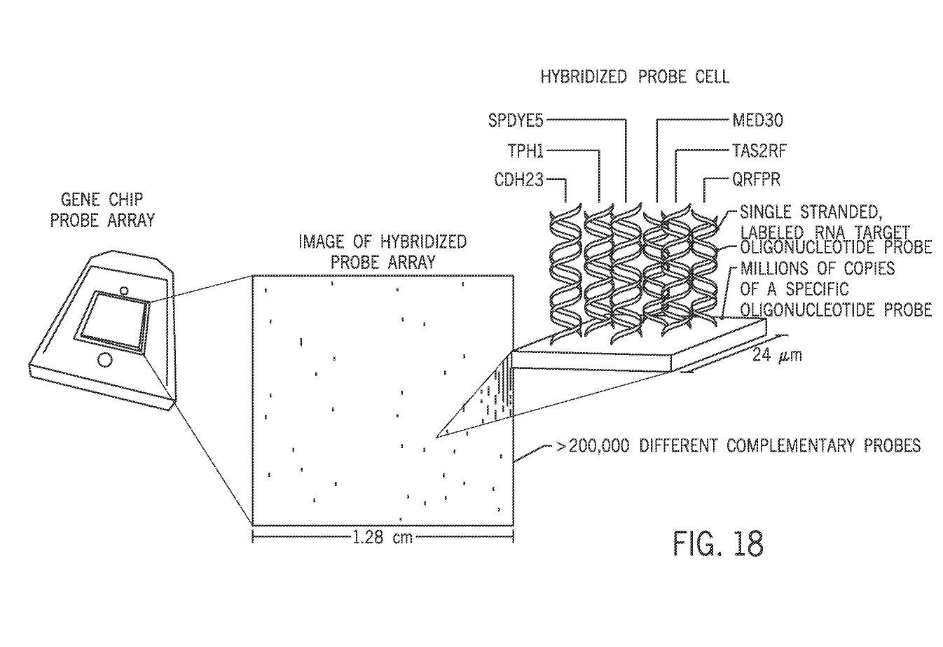

As those skilled in the art will appreciate and referring to FIG. 18, a DNA microarray (also commonly known as DNA chip or biochip) is a collection of microscopic DNA spots attached to a solid surface. DNA microarrays can be used to measure the expression levels of large numbers of genes simultaneously. Each DNA spot contains picomoles (10⁻¹² moles) of a specific DNA sequence, known as probes. These can be a short section of a gene or other DNA element that are used to hybridize a cDNA or cRNA (also called anti-sense RNA) sample (called target) under high-stringency conditions. Probe-target hybridization is usually detected and quantified by detection of fluorophore-, silver-, or chemiluminescence-labeled targets to determine relative abundance of nucleic acid sequences in the target.

Applicants' invention was developed in accordance with the protocol and research consents approved by the University of Arizona College of Medicine Institutional Review Board. Brain tissue samples were obtained from subjects who underwent ATL/AH at the University of Arizona Health Network (Tucson, Ariz.). All tissue samples were stored in RNAlater® RNA stabilization Solution ((liagen, Valencia, Calif., USA) at −80° C. until RNA extraction was performed. RNA was extracted using the RNeasy lipid tissue mini kit (Qiagen, Valencia, Calif., USA) following manufacturer's instructions. RNA was then stored at −80° C. until RNA analysis was performed.

RNA A260/A280 was 1.3 to 2.1 and yield was 3.7 to 30.2 μg from 100 mg of brain tissue. The density plot showed no outlying arrays. Positive and Negative control probes showed good separation in expression values. Background and non-background corrected values were similar. Overall these arrays had consistently high quality data.

Nineteen (19) patients underwent ATL/AH for medically intractable temporal lobe seizures. All patients had lateral temporal cortex removed for genetic analysis. These subjects were dichotomized into two groups, post-operatively seizure-free (N=11) or non-seizure-free post ATL/AH (N=8). The mean age for the seizure-free group was 37.1±10.6 years. The mean age for the non-seizure-free group was 37.3±9.7 years (p=0.97). There were five (5) males in both groups, the seizure-free group has six (6) females and the non-seizure-free group had three (3) females (p=0.463).

In the seizure-free group there were seven (7) white, three (3) Hispanic and one (1) Native American/Eskimo subjects. In the non-seizure-free positive group there were three (3) white, three (3) Hispanic and two (2) Native American/Eskimo subjects (p=0.47). The mean post operative follow up was 30.5±23.5 months for the seizure free group and 43.6±19.1 months for the non-seizure free group (p=0.21).

The isolated total RNA samples were used to produce labeled target, hybridized to Affymetrix GeneChip® Human Gene 1.0 ST Array, and read using the Agilent/Affymetrix 2500A scanner according to manufacturer's protocols.

Biological process pathways in medically intractable temporal lobe epilepsy patients were analyzed for significant deviation from normal. Whole genome and targeted gene expression data were analyzed for prognostic value in predicting seizure-free outcome following ATL/AH.

Demographic statistics were conducted using IBM SPSS statistic20 (version 20, IBM corporation, Armonk, N.Y.). Comparisons were made using X2 analysis for gender and race. Student t-test was used to analyze the significance of age among the groups. The level of significance was established at 0.05.

Gene analysis was performed utilizing the bioinformatics and statistical tools provided by the R programming and BioConductor projects (www.rproject.org and www.bioconductor.org). The analysis included assessment of data quality, positive and negative controls on the Affymetrix ST1.0 microarrays, probe annotation, and analysis of pathways and biological function (gene ontologies). The statistical analysis between seizure-free and non-seizure-free groups was performed using the limma library. Whole genome data were analyzed for prognostic value for seizure-free outcome following ATL/AH by logistic regression.

Logistic regression analysis identified ten genes that met criteria for having significantly different expression levels between seizure free and non-seizure free groups (auc>0.9 & p<0.05). Seizure free subjects had a significant relative down-regulation of speedy homolog E5 (Xenopus laevis)(SPDYE5) (p=0.0256, AUC=0.955); tryptophan hydroxylase 1(TPH1), (p=0.0367, AUC=0.92); cadherin-related 23(CDH23) (p=0.0309, AUC=0.943); taste receptor, type 2, member 5 (TAS2R5) (p=0.0272, AUC=0.92) and the seizure free groups had a relative up-regulation of mediator complex subunit 30 (MED30) (p=0.0376 AUC=0.909); and pyroglutamylated RF amide peptide receptor (QRFPR), (p=0.0471, AUC=0.955).

Pathway analysis was performed using GO and KEGG terms. Gene Ontology allows researchers to consistently describe gene products, while KEGG terms describe genomes, enzymatic pathways and biological chemicals. There were significant differences between seizure-free and non-seizure-free groups in 6 pathways.

The Gene Ontology (GO) and Kyoto encyclopedia of Genes and Genomes (KEGG) databases were used to identify global trends in gene expression data within this invention. This invention describes differences in expression between the seizure-free and non-seizure-free group pathways of 3 GO terms and 3 KEGG terms.

Cytoskeletal anchoring at nuclear membrane (GO:0090286) & SUN-KASH complex (GO:0034993). This invention describes differences in up-regulation seen in these two pathways associated with the non-seizure free subjects. These pathways represent biological processes and cellular components.

The cytoskeletal anchoring at nuclear membraneGO term is defined as “the process in which cytoskeletal filaments are directly or indirectly linked to the nuclear membrane” 51. The SUN-KASH complex is defined as “a protein complex that spans the nuclear outer and inner membranes, thereby linking the major cytoplasmic cytoskeleton elements to the nuclear lumen; the complex is conserved in eukaryotes and contains proteins with SUN and KASH domains”.

While these two terms are representative of two different Go terms, they include the same 5 genes. Two of these genes showed significant differences in gene expression. These two genes were spectrin repeat containing, nuclear envelope family member 3 (SYNE3 aka C14orf49) and Sad1 and UNC84 domain containing 1 (SUN1). SYNE3 is also known as Nesprin-3 and has been shown to regulate vascular endothelial cell shape, perinuclear cytoskeletal architecture, and important aspects of flow mediated mechanotransduction. The SUN1 protein is involved in nuclear anchorage and migration. In addition it has been associated with laminopathies, and hearing. Neither gene has been previously reported to be involved with epilepsy.

DNA methylation involved in gamete generation (GO:0043046). This invention demonstrates that the non-seizure-free group was associated with an up regulation of the DNA methylation involved in gamete generation. This pathway represents a biological process pathway. It is defined as “The covalent transfer of a methyl group to C-5 of cytosine that contributes to the establishment of DNA methylation patterns in the gamete”.

There were 2 genes out of 10 that showed differences in expression these were CCCTC-binding factor (zinc finger protein)-like (CTCFL) and DNA (cytosine-5-)-methyltransferase 3 alpha (DNMT3A). CTCFL and DNMT3A are involved with DNA methylation during embryo development. Both genes have been associated with numerous types of cancer. It has been shown in a rat model that an increase in hippocampal DNA methylation correlates with increased DNA methyltransferase activity, disruption of adenosine homeostasis and spontaneous recurrent seizures (60a). Furthermore, pathological changes in DNA methylation homeostasis have been suggested to underlie epileptogenesis. Reversal of these epigenetic changes with adenosine augmentation therapy have been shown to halt disease progression. This is the first study to find an association between temporal cortical DNA methylation involved in gamete generation (GO:0043046) with continued seizures after ATL/AH.

Phototransduction (KEGG:0474). This invention establishes that the phototransduction pathway was associated with up regulation in the non-seizure-free subjects. This pathway is described as follows: “Phototransduction is a biochemical process by which the photoreceptor cells generate electrical signals in response to captured photons. The vertebrate cascade starts with the absorption of photons by the photoreceptive pigments, the rhodopsins, which consist of a membrane embedded chromophore, 11-cis-retinal, and a G-protein-coupled receptor, opsin. The photon isomerizes 11-cis-retinal to all-trans-retinal which induces a structural change that activates the opsin. This triggers hydrolysis of cGMP by activating a transducinphosphodiesterase 6 (PDE6) cascade, which results in closure of the cGMP-gated cation channels (CNG) in the plasma membrane and membrane hyperpolarization. The hyperpolarization of the membrane potential of the photoreceptor cell modulates the release of neurotransmitters to downstream cells. Recovery from light involves the deactivation of the light-activated intermediates: photolyzed rhodopsin is phosphorylated by rhodopsin kinase (RK) and subsequently capped off by arrestin; GTP-binding transducin alpha subunit deactivates through a process that is stimulated by RGS9.”

The two genes associated with this pathway that showed differences in gene expression are solute carrier family 24 (sodium/potassium/calcium exchanger), member 1 (SLC24A1) and guanylate cyclase activator 1C (GUCA1C). Most recently, a mutation of the SLC24A1 gene has been associated with night blindness 63 and other retinal diseases 64. In addition, the SLC24A1 gene has been associated with mean corpuscular hemoglobin in a genome-wide study 65. Autosomal dominant cone dystrophies have been associated with mutations of GUCA1C.

Drug metabolism-other enzymes (KEGG:00983). This invention establishes that the drug metabolism-other enzymes pathway was associated with down regulation in the non-seizure free subjects. This pathway is involved in biodegradation and metabolism of xenobiotics 61, 62. The genes that showed differences in gene expression were uridine diphospho glucuronosyltransferase 2 family, polypeptide B17 (UGT2B17) and carboxylesterase 1 (CES1). UGT2B17 may be the major enzyme responsible for the metabolism of drugs such as MK-7246, nicotine 8, and steroid hormones. This gene has also been associated with several types of cancer. The CES1 gene has been associated with obesity, cardiovascular risk factors in obesity and changes blood brain barrier permeability to some drugs.

Arginine and proline metabolism (KEGG:00330). In this invention, this pathway is also associated with down regulation in the non-seizure free subjects and is involved in amino acid metabolism. The two genes that showed differences in gene expression were aldehyde dehydrogenase 4 family, member A1 (ALDH4A1) and spermidine synthase (SRM). ALDH4A1 has been associated with type II hyperporlinemia which is an autosomal recessive disorder. Finally, the SRM gene is a mediator of cell growth and differentiation.

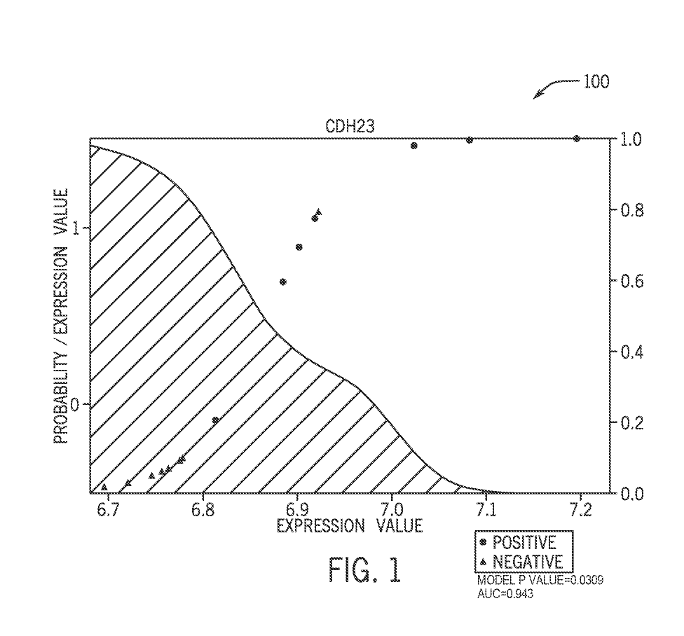

Targeted gene expression analysis detected significant downregulation for CDH23, cadherin-related 23, model p value=0.0309, AUC=0.943. FIG. 1 illustrates a conditional probability plot for gene CDH23 which meets criteria of area under receiver operating characteristic curve>0.90 and p value<0.05. Positive (red) points represent patients where seizures persisted post surgery. Negative (blue) points represent patients who were rendered seizure-free by ATL/AH.

Relative down-regulation of cadherin-related 23(CDH23) temporal cortical gene expression was associated with seizure-free outcome following ATL/AH. The Cadherin-related 23(CDH23) gene is located on chromosome 10q22.1. A member of the cadherin superfamily, this gene encodes for calcium dependent cell-cell adhesion glycoproteins. The encoded protein is involved in stereocilia organization and hair bundle formation and is located in a region containing two human deafness loci. Polymorphisms of this gene have been associated with hearing loss, Usher Syndrome (sensorineural deafness and retinitis pigmentosa), anchoring of stereocilia, and breast cancer. Polymorphisms of protocadherin 19 (PCDH19) have been associated with epilepsy. PCDH19 is also in the cadherin superfamily and is primarily expressed in the brain. Auditory auras and fluctuating hearing loss and acute transient deafness, both presumably due to primary auditory cortical dysfunction, have been associated with temporal lobe epilepsy. While the specific mechanism is unknown these two genes may have similar mechanisms in regards to epilepsy and seizure presence.

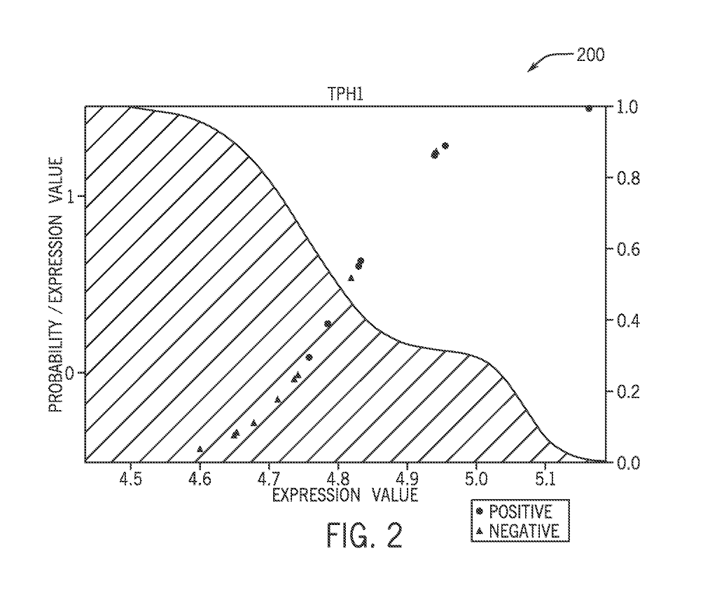

Targeted gene expression analysis detected significant downregulation for TPH1, tryptophan hydroxylase 1, model p value=0.0367, AUC=0.92. FIG. 2 illustrates conditional probability plot 200 for gene TPH1, wherein plot 200 meets criteria of area under receiver operating characteristic curve>0.90 and p value<0.05. Positive (red) points represent patients where seizures persisted post surgery. Negative (blue) points represent patients who were rendered seizure-free by ATL/AH.

Relative down-regulation of tryptophan hydroxylase 1 (TPH1) temporal cortical gene expression was associated with seizure-free outcome following ATL/AH. The TPH1 gene is located on chromosome 11p15.3-p14 and encodes a member of the aromatic amino acid hydroxylase family. Tryptophan hydroxylase is the rate-limiting enzyme in the biosynthesis of serotonin (5-HT). TPH1 polymorphisms have been associated with numerous diseases including schizophrenia, bipolar disorder, mood disorder associated with suicide, and other brain based diseases. TPH abnormalities have been reported in animal models of epilepsy. In mice, the primary action of an audiogenic seizure-inducing catecholamide, H13/04, is to decrease the synthesis rate of 5-HT by competitive inhibition of tryptophan hydroxylase. Similarly, TPH1 levels in mice susceptible to audiogenic seizures were significantly lower in brains when compared to normal mice. Statnick and associates' findings showed a reduction in tryptophan hydroxylase in the brains of genetically epilepsy prone rats (GEPR-9s). While these findings are counterintuitive to our findings, it still supports TPH1's possible role in seizure status following ATL/AH.

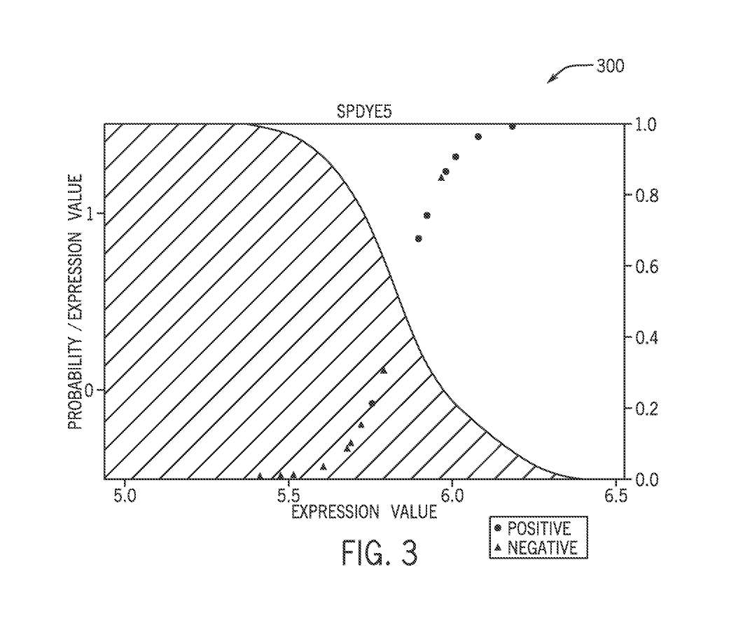

Targeted gene expression analysis detected significant downregulation for SPDYE5, speedy homolog E5, model p value=0.0256, AUC=0.955. FIG. 3 illustrates a conditional probability plot 300 for gene SPDYE5, wherein plot 300 meets criteria of area under receiver operating characteristic curve>0.90 and p value<0.05. Positive (red) points represent patients where seizures persisted post surgery. Negative (blue) points represent patients who were rendered seizure-free by ATL/AH.

Relative down-regulation of speedy homolog E5 (SPDYE5) temporal cortical gene expression was associated with seizure-free outcome following ATL/AH. SPDYE5 is located on chromosome 7q11.23. It was first sequenced in 2003. While no additional literature appears for the SPDYE5 gene, chromosome 7q11.23 abnormalities, specifically gene deletions, have been associated with intellectual disabilities, epilepsy and neurobehavioral problems. Chromosome 7q11.23 contains the region responsible for Williams-Beuren syndrome caused by a hemizygous contiguous gene deletion producing symptoms of supravalvular aortic stenosis, mental retardation, overfriendliness and visuaspatial impairment. Duplication of 7q11.23, reciprocal of the Williams-Beuren deletion, has been reported in one case of left temporal lobe cortical dysplasia and epilepsy. Deletion of one gene within 7q11.23 has also been associated with infantile spasms.

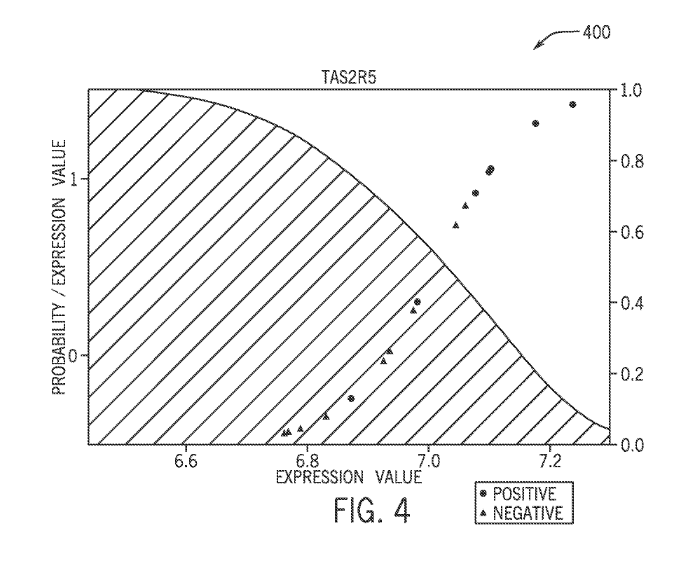

Targeted gene expression analysis detected significant downregulation for TAS2R5, taste receptor, type 2, member 5, model p value=0.0272, AUC=0.92). FIG. 4 illustrates a conditional probability plot 400 for gene TAS2R5, wherein plot 400 meets criteria of area under receiver operating characteristic curve>0.90 and p value<0.05. Positive (red) points represent patients where seizures persisted post surgery. Negative (blue) points represent patients who were rendered seizure-free by ATL/AH.

Relative down-regulation of the taste receptor, type 2, member 5 (TAS2R5) temporal cortical gene expression was associated with seizure-free outcome following ATL/AH. The TAS2R5 gene is located on chromosome 7q31.3-q32 and encodes a bitter taste receptor, a member of the G protein-coupled receptor superfamily specifically expressed by taste receptor cells of the tongue and palate epithelia⁴⁰. While the literature on this taste receptor is relatively small, taste sensitivity has been associated with epilepsy. Pal and associates investigated phenylthiocarbamide (PTC) taste sensitivity, which is a genetically controlled trait. The ability to taste the PTC allele is dominant over the non-taster allele. Those people that are non-tasters tend to ingest a greater quantity of bitter tasting goitrogenic substances present in naturally edible plants. It has been postulated that non-tasters may have greater thyroid stress during development which may make them more susceptible to epilepsy than people that can taste PTC.

In addition, the literature supports the role of the anterior temporal lobe in taste perception. The anterior temporal lobe has been shown to have important function involving low-level gustatory perception Patients undergoing right temporal lobe resection for intractable temporal lobe epilepsy have rated the bitter taste as more intense than patients undergoing left temporal lobectomy or healthy controls. Functional neuroimaging study has shown activation to stimulation of aversive taste in the left amygdala, resection of which for treatment of intractable temporal lobe epilepsy produced gustatory agnosia, including the inability to recognize bitter taste. Resection of the right anteromedial temporal lobe in humans has been reported to result in increased aversive bitter taste perception.

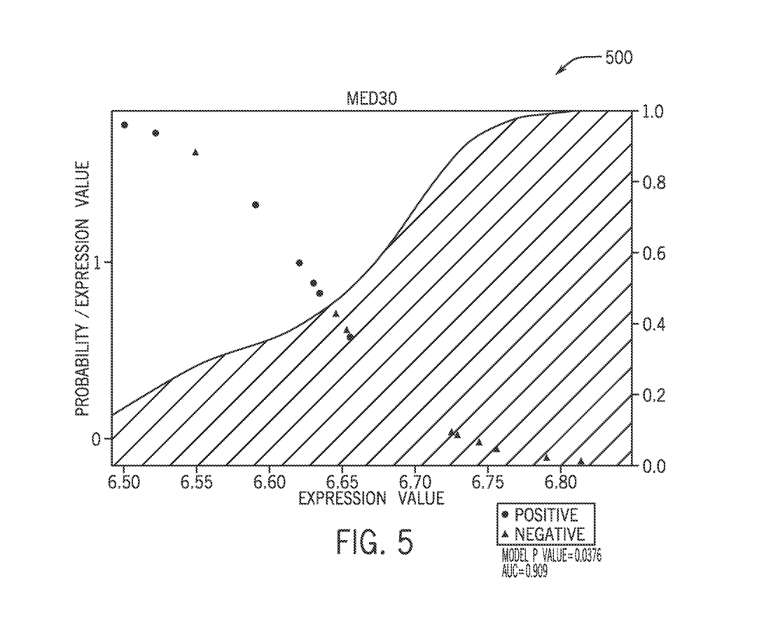

Targeted gene expression analysis detected significant upregulation for MED30, mediator complex subunit 30, model p value=0.0376, AUC=0.909. FIG. 5 illustrates a conditional probability plot 500 for gene MED30, wherein plot 500 meets criteria of area under receiver operating characteristic curve>0.90 and p value<0.05. Positive (red) points represent patients where seizures persisted post surgery. Negative (blue) points represent patients who were rendered seizure-free by ATL/AH.

Relative up-regulation of mediator complex subunit 30 (MED30) temporal cortical gene expression was associated with seizure-free outcome following ATL/AH. The MED30 gene is located on chromosome 8q24.11 and facilitates gene expression through a wide variety of metazoan specific transcriptional activators. Mediator of RNA polymerase II transcription subunits (MEDs) promote the assembly, activation and regeneration of transcription complexes on core promoters during the initiation and re-initiation phases of transcription. There are numerous mediator complex subunits.

While this is the first time that MED30 has been associated with epilepsy, other mediator complex subunits have been associated with neurodevelopmental disorders. These disorders include Charcot-Marie-Tooth (CMT) disease, infantile cerebral and cerebellar atrophy and syndromal X-linked mental retardation. Influence of the MED30 gene on gene expression may be a key to understanding seizure continuation following ATL/AH.

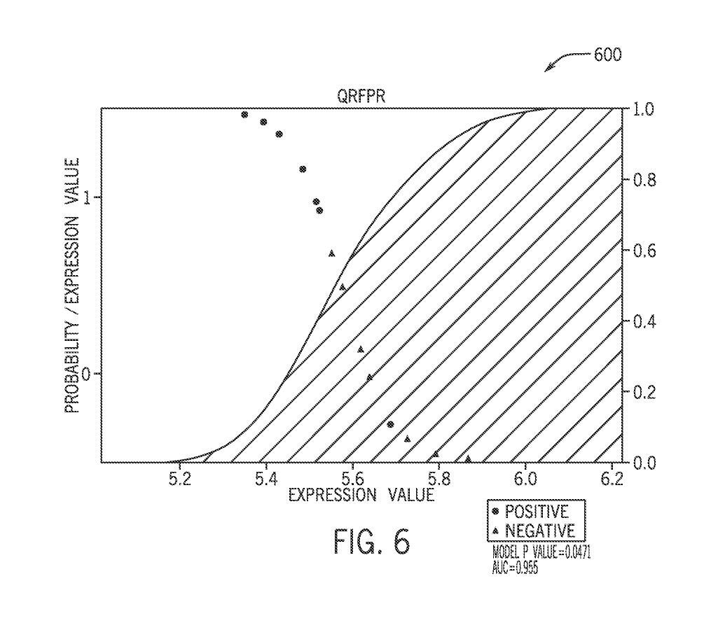

Targeted gene expression analysis further detected significant upregulation for QRFPR, pyroglutamylated RF amide peptide receptor, model p value=0.0471, AUC=0.955). FIG. 6 illustrates a conditional probability plot 600 for gene QRFPR, wherein plot 600 meets criteria of area under receiver operating characteristic curve>0.90 and p value<0.05. Positive (red) points represent patients where seizures persisted post surgery. Negative (blue) points represent patients who were rendered seizure-free by ATL/AH.

Relative up-regulation of pyroglutamylated RF amide peptide receptor (QRFPR) temporal cortical gene expression was associated with seizure-free outcome following ATL/AH. The QRFPR gene is located on chromosome 4q27. This is a receptor for the orexigenic neuropeptide QRFP. The activity of this receptor is mediated by G proteins that modulate adenylate cyclase activity and intracellular calcium levels. In mice, the activation of G protein-coupled receptor 103 (GPR103) by its endogenous peptidic ligands, QRFPs, is involved in the central regulation of feeding by increasing food intake, body weight and fat mass. QRFP is bioactive in omental adipocytes from obese individuals, inhibiting isoproterenol (ISO)-induced lipolysis in these cells 49. GPR103b and QRFP work in an autocrine/paracrine manner to regulate adipogenesis.

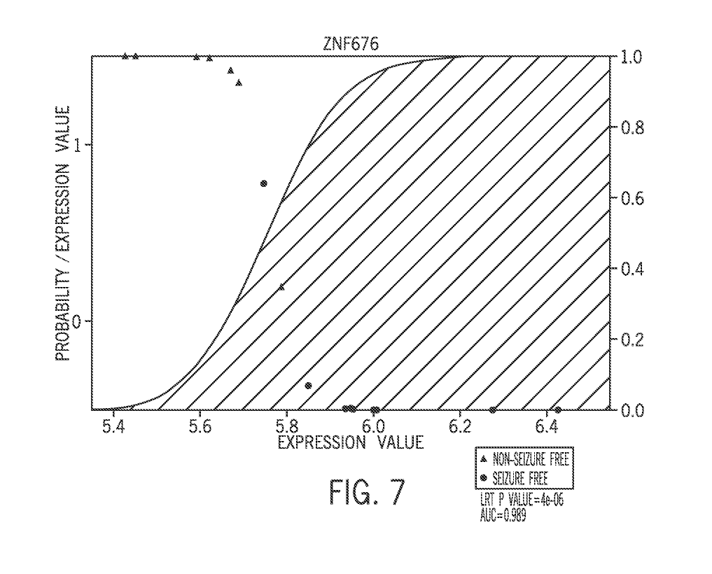

Targeted gene expression analysis has also detected significant relative up-regulation of ZNF676 zinc finger protein 676 (model p value=0.000004, auc=0.989). FIG. 7 illustrates a conditional probability plot 500 for gene ZNF676, wherein FIG. 7 meets criteria of area under receiver operating characteristic curve>0.90 and p value<0.05. Positive (red) points represent patients where seizures persisted post surgery. Negative (blue) points represent patients who were rendered seizure-free by ATL/AH.

Relative up-regulation of ZNF676 zinc finger protein 676 temporal cortical gene expression was associated with seizure-free outcome following ATL/AH. The ZNF676 gene is located on chromosome 19p12. A novel genomic region mapped near zinc finger protein 676 regulates leukocyte telomere length (LTL) which is associated with a number of common age-related diseases and which is a heritable trait. This gene has also been identified as a potential biomarker for detecting deficiency in the iodothyronine deiodinase family of selenoenzymes which activate and inactivate thyroid hormones through the removal of specific iodine moieties from thyroxine and its derivatives (80). The expression of activating and inactivating deiodinases plays a critical role in neuronal cell systems during development as well as in adult vertebrates (80). The prognostic value of up-regulated temporal cortical zinc finger protein 676 gene expression for post-operative ATL/AH seizure-free outcome is the first association reported between the ZNF676 gene and epilepsy.

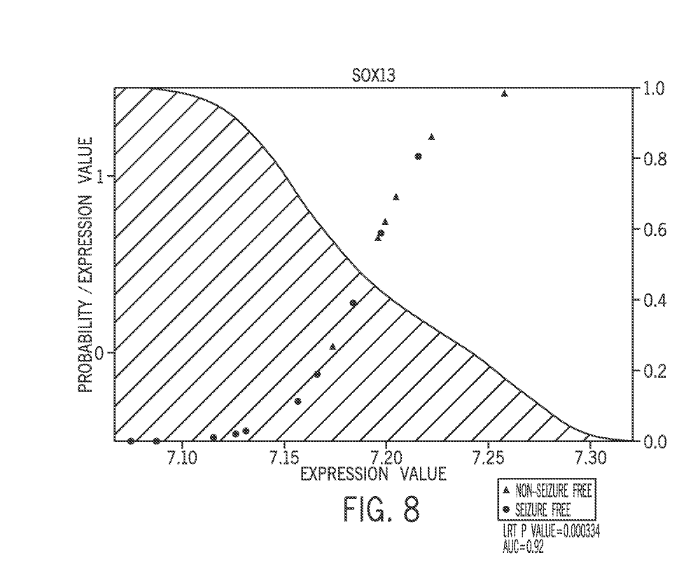

Targeted gene expression analysis has also detected significant down-regulation of SOX13 SRY (sex determining region Y)-box 13 (model p value=0.000334, auc=0.920). FIG. 8 illustrates a conditional probability plot 500 for gene SOX13 SRY, wherein FIG. 8 meets criteria of area under receiver operating characteristic curve>0.90 and p value<0.05. Positive (red) points represent patients where seizures persisted post surgery. Negative (blue) points represent patients who were rendered seizure-free by ATL/AH.

Relative down-regulation of SOX13 SRY (sex determining region Y)-box 13 temporal cortical gene expression was associated with seizure-free outcome following ATL/AH. The SOX13 gene is located on chromosome 1q32. This gene encodes the SOX (SRY-related HMG-box) transcription factor family which is involved in regulation of embryogenesis (84) and the determination of cell fate. Expressed in three embryonic cell lineages, SOX13 may direct various developmental processes. Consistent with a role for SOX13 as a transcription factor during neurogenesis, the SOX13 protein localizes to the nuclei of the differentiating neuronal cells. During neurogenesis, SOX13 expression is activated in a subset of neural progenitors during exit from the mitotic cycle and migration from the ventricular zone.

SOX13 is also expressed in the developing neural tube and in the condensing mesenchyme and cartilage progenitor cells during limb endochondral bone formation and in the somite sclerotome and its derivatives. In addition, SOX13 is detected in the developing kidney, pancreas and liver and in the visceral mesoderm of the extra-embryonic yolk sac and spongiotrophoblast layer of the placenta. The gene is also a type-1 diabetes autoantigen, known as islet cell antibody 12. The SOX13 gene is upregulated in oligodendrogliomas. The prognostic value of relative down-regulated temporal cortical SOX13 SRY (sex determining region Y)-box 13 gene expression for post-operative ATL/AH seizure-free outcome is the first association reported between the SOX13 gene and epilepsy.

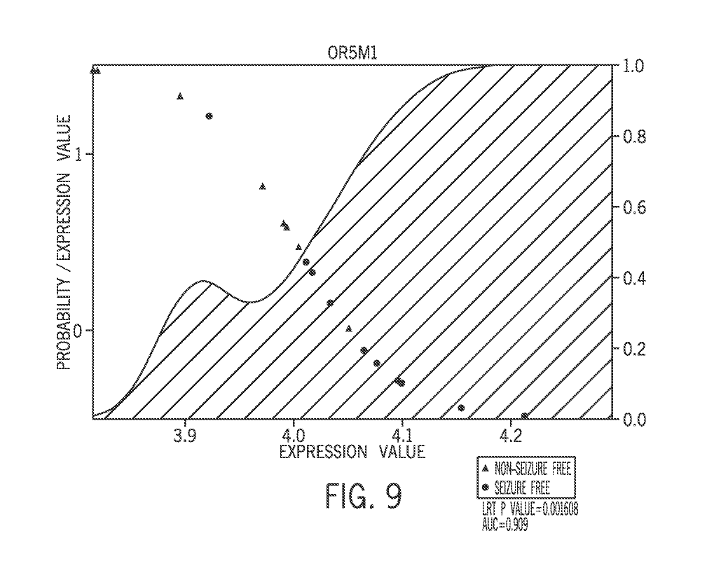

Targeted gene expression analysis has also detected significant up-regulation of OR5M1 olfactory receptor, family 5, subfamily M, member 1 (model p value=0.001608, auc=0.909). FIG. 9 illustrates a conditional probability plot 500 for gene OR5M1, wherein FIG. 9 meets criteria of area under receiver operating characteristic curve>0.90 and p value<0.05. Positive (red) points represent patients where seizures persisted post surgery. Negative (blue) points represent patients who were rendered seizure-free by ATL/AH.

Relative up-regulation of OR5M1 olfactory receptor, family 5, subfamily M, member 1 temporal cortical gene expression was associated with seizure-free outcome following ATL/AH. The OR5M1 gene is located on chromosome 11q12.1. The olfactory receptor gene family is the largest in the genome.

Single coding-exon genes give rise to olfactory receptor proteins which are members of a large family of G-protein-coupled receptors (GPCR) (85). A 7-transmembrane domain structure is shared by olfactory receptors with many neurotransmitter and hormone receptors. Olfactory receptors recognize and G-proteins mediate odorant signal transduction. Odorants detected by the large family of olfactory receptors and expressed by a small subset of olfactory sensory neurons can induce seizures.

The temporal lobe plays a crucial role in olfaction and olfactory function may be disturbed in temporal lobe epilepsy. Olfactory hallucinations may be an aura of temporal lobe epilepsy and electrical stimulation of medial temporal lobe structures, including the amygdala, may induce olfactory sensations. Functional disintegration of olfactory stimuli with failure to activate the amygdala in the epileptic hemisphere has been detected with cerebral blood flow PET in medial temporal lobe epilepsy patients.

Impaired higher order processing of olfactory sensations in temporal lobe epilepsy (TLE) has been suggested as the explanation for that fact that olfactory stimulants which affect cognition in healthy subjects have limited effects in TLE. In the current invention, the prognostic value of up-regulated temporal cortical OR5M1 olfactory receptor, family 5, subfamily M, member 1 gene expression for post-operative ATL/AH seizure-free outcome is the first association reported between the OR5M1 olfactory receptor gene and epilepsy.

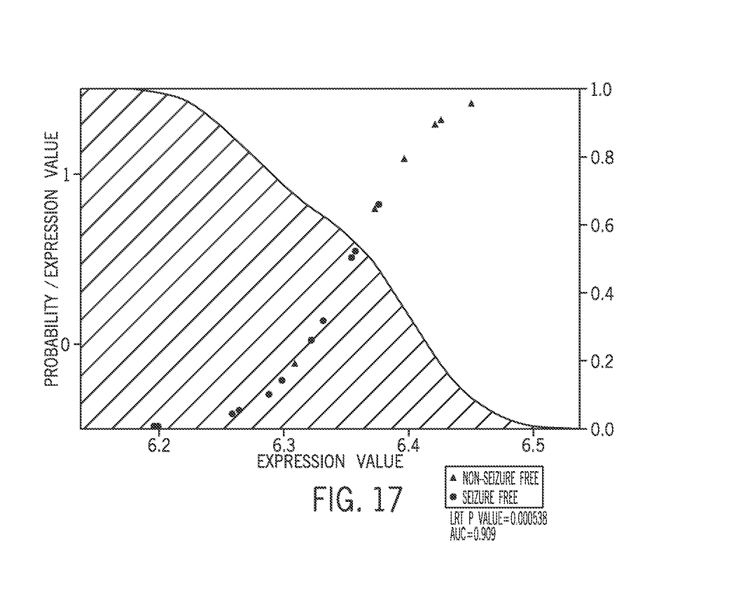

Targeted gene expression analysis has also detected significant down-regulation of Selenophosphate Synthetase 1 Pseudogene 1 (SEPHS1P) (model p value=0.000538, auc=0.909). FIG. 17 illustrates a conditional probability plot for gene SEPHS1P, wherein FIG. 17 meets criteria of area under receiver operating characteristic curve>0.90 and p value<0.05. Positive (red) points represent patients where seizures persisted post surgery. Negative (blue) points represent patients who were rendered seizure-free by ATL/AH.

Relative down-regulation of Selenophosphate Synthetase 1 Pseudogene 1 (SEPHS1P) temporal cortical gene expression is associated with seizure-free outcome following ATL/AH.

SEPHS1P is located on chromosome 7q11.21. The Reference Sequence database of the National Center for Biotechnology Information (NCBI) indicates that this gene has been, heretofore, considered as a non-transcribed pseudogene and is listed as non-coding RNA.

GeneCards lists the Selenophosphate Synthetase 1 Pseudogene 1 (SEPHS1P) as an alias for this locus (External Ids: HGNC: 421611, Entrez Gene: 1684742, Ensembl: ENSG000001827227) (92,93) The National Center for Biotechnology Information, NCBI, lists SEPHS1P1 selenophosphate synthetase 1 pseudogene 1 [Homo sapiens human)] under this heading: Gene ID: 168474, updated on 26 Feb. 2013.

Pseudogenes are generally considered dysfunctional relatives of genes which have lost their protein-coding ability or which may no longer be expressed within the cell. Pseudogenes may result from accumulation of mutations within an organism's non-essential genome. However, most pseudogenes have some gene-like features such as promoters, CpG islands and splice sites. Considered non-functional due to the lack of protein-coding ability, pseudogenes are often labeled as “junk DNA”.

In the current invention, the prognostic value of down-regulation of Selenophosphate Synthetase 1 Pseudogene 1 (SEPHS1P) temporal cortical expression for seizure-free outcome following ATL/AH is the first reported function of SEPHS1P.

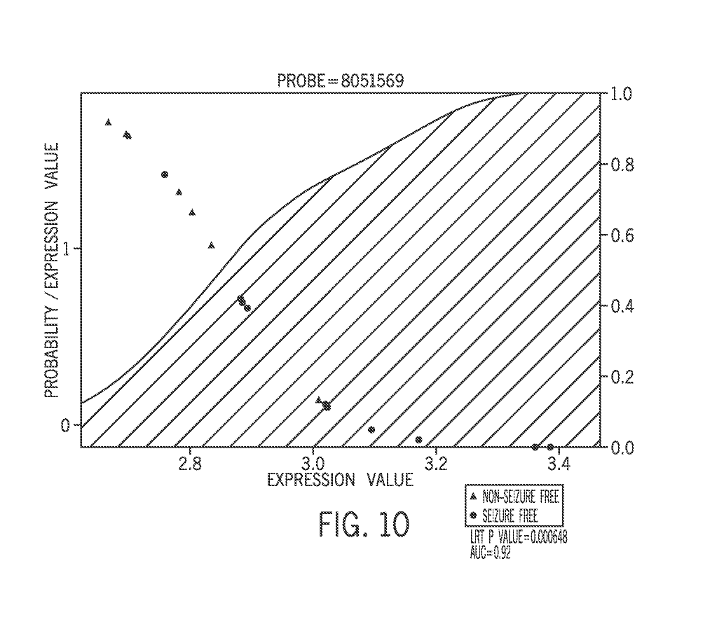

Targeted gene expression analysis has also detected significant up-regulation of RNA Probe rs8051569 associated with seizure-free outcome following ATL/AH. FIG. 10 illustrates a conditional probability plot for RNA Probe rs8051569, wherein FIG. 10 meets criteria of area under receiver operating characteristic curve>0.90 and p value<0.05. Positive (red) points represent patients where seizures persisted post surgery. Negative (blue) points represent patients who were rendered seizure-free by ATL/AH.

rs8051569 is a human single nucleotide polymorphism (SNP) involving substitution of cytosine for adenine on chromosome 16q.23 (22a,b). The National Center for Biotechnology Information (NCBI) maintains an SNP database (dbSNP) in which SNPs are referred to by their dbSNP “rs number”, as with this RNA probe.

Heretofore, this human chromosome 16 single nucleotide polymorphism had no known clinical significance. The prognostic value of up-regulation of temporal cortical rs8051569 expression for seizure-free outcome following ATL/AH is the first reported association between this SNP and epilepsy.

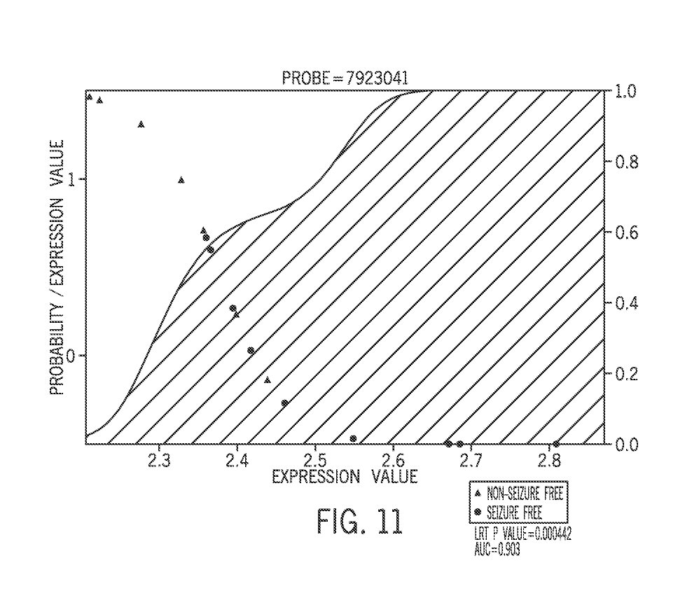

Targeted gene expression analysis has also detected significant up-regulation of RNA Probe rs7923041 (now merged into rs1775436) associated with seizure-free outcome following ATL/AH. FIG. 11 illustrates a conditional probability plot for RNA Probe rs7923041, wherein FIG. 11 meets criteria of area under receiver operating characteristic curve>0.90 and p value<0.05. Positive (red) points represent patients where seizures persisted post surgery. Negative (blue) points represent patients who were rendered seizure-free by ATL/AH. rs7923041 is a human single nucleotide polymorphism (SNP) located on chromosome 10p11.2. Heretofore, this human chromosome 10 single nucleotide polymorphism had no known clinical significance. The prognostic value of relative up-regulation of rs7923041 for seizure-free outcome following ATL/AH is the first reported association between this SNP and epilepsy.

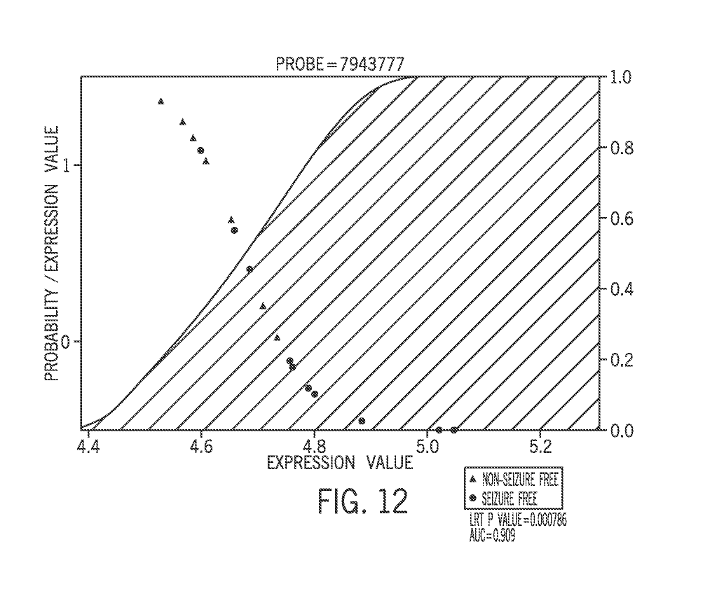

Targeted gene expression analysis has also detected significant up-regulation of the RNA probe, rs7943777 associated with seizure-free outcome following ATL/AH. FIG. 12 illustrates a conditional probability plot for RNA Probe rs7943777, wherein FIG. 12 meets criteria of area under receiver operating characteristic curve>0.90 and p value<0.05. Positive (red) points represent patients where seizures persisted post surgery. Negative (blue) points represent patients who were rendered seizure-free by ATL/AH. The RNA Probe rs7943777 is a single human nucleotide polymorphism (SNP) of chromosome 11 substituting thymine for cytosine in the ancestral allele. This SNP is located in intron NM_152432.2, known as Rho GTPase Activating Protein 42, which is located on chromosome 11q22.1. GTPase-activating proteins enhance intrinsic GTPase activity, leading to the inactive state of GTPase. Operating as molecular switches coupling changes in the external environment to intracellular signal-transduction pathways, Rho GTPases regulate cellular activities including cell dynamics, cell growth, intracellular membrane trafficking, gene transcription, cell-cycle progression and apoptosis. Heretofore, this human chromosome 11 single nucleotide polymorphism had no known clinical significance. The prognostic value of relative up-regulation of rs7943777, for seizure-free outcome following ATL/AH is the first reported association between this SNP probe and epilepsy.

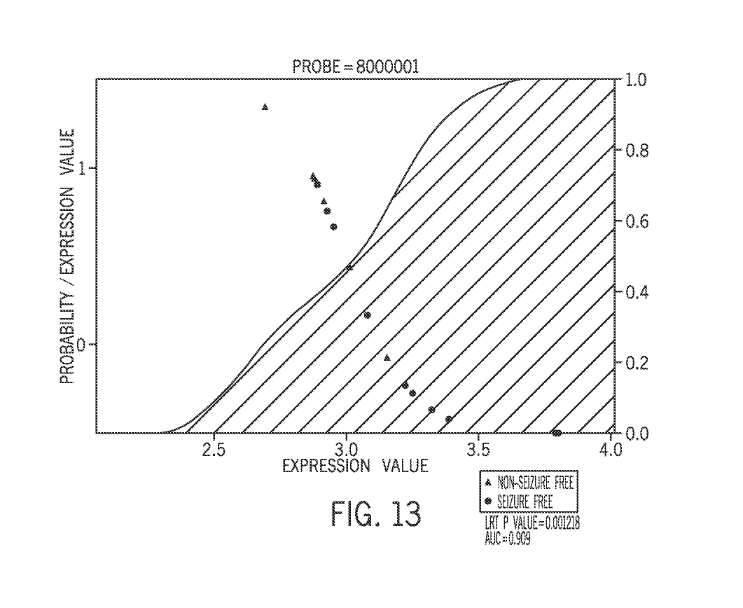

Targeted gene expression analysis has also detected significant up-regulation of the RNA probe, rs8000001 associated with seizure-free outcome following ATL/AH. FIG. 13 illustrates a conditional probability plot for RNA Probe rs8000001, wherein FIG. 13 meets criteria of area under receiver operating characteristic curve>0.90 and p value<0.05. Positive (red) points represent patients where seizures persisted post surgery. Negative (blue) points represent patients who were rendered seizure-free by ATL/AH. The RNA Probe rs8000001 is a single human nucleotide polymorphism (SNP) of chromosome 13q21.1 substituting adenine for guanine in the ancestral allele. The most proximal breakpoint ever detected in a ring chromosome 13 disorder has been reported at 13q21.1 in association with anencephaly, cranial bone defect at the vault and other malformations. A variant subtype of the late-infantile form of human neuronal ceroid lipofuscinoses (NCL), an autosomal recessive progressive encephalopathy including seizures and the accumulation of ceroid-and lipofuscin-like cytosomes in neural and extraneural tissues, is also mapped to a well-defined region including 13q21.1. Heretofore, this single nucleotide polymorphism had no known clinical significance. The prognostic value of relative up-regulation of rs8000001 for seizure-free outcome following ATL/AH is the first reported association between this SNP and epilepsy.

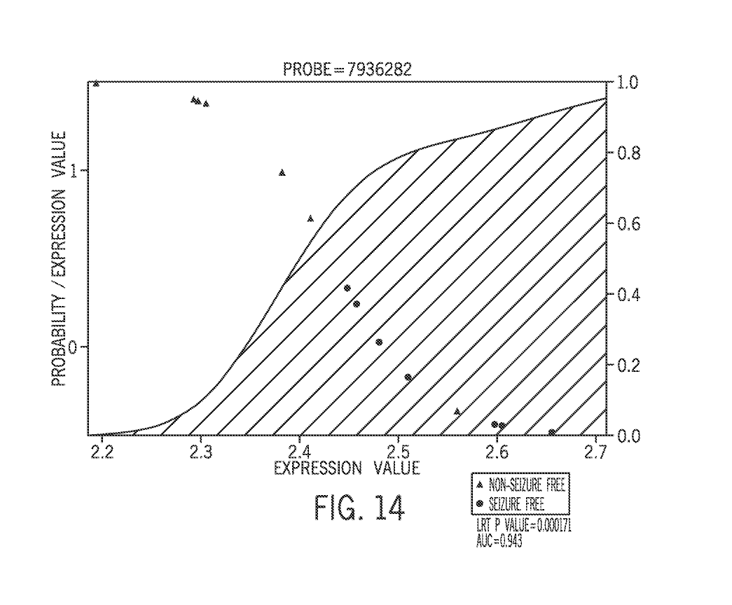

Targeted gene expression analysis has also detected significant down-regulation of RNA probe rs7936282 associated with seizure-free outcome following ATL/AH. FIG. 14 illustrates a conditional probability plot for Probe rs7936282, wherein FIG. 14 meets criteria of area under receiver operating characteristic curve>0.90 and p value<0.05. Positive (red) points represent patients where seizures persisted post surgery. Negative (blue) points represent patients who were rendered seizure-free by ATL/AH. rs7936282 is a single nucleotide polymorphism (SNP) in which cytosine is substituted for adenine on chromosome 11q22.1. Heretofore, the clinical significance of rs7936282 has been unknown. The prognostic value of down-regulation of temporal cortical rs7936282 expression for seizure-free outcome following ATL/AH is the first reported association between this SNP and epilepsy.

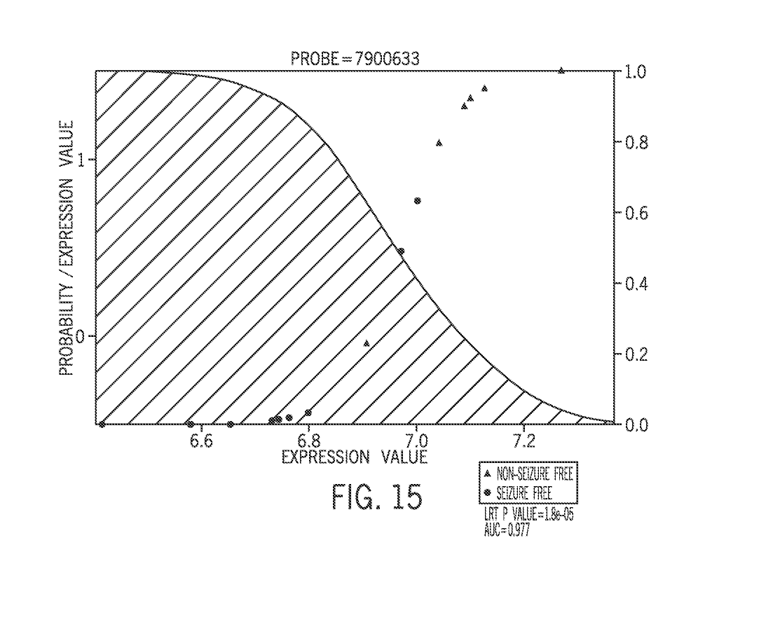

Targeted gene expression analysis has also detected significant down-regulation of temporal cortical rs7900633 expression associated with seizure-free outcome following ATL/AH. FIG. 15 illustrates a conditional probability plot for RNA Probe rs7900633, wherein FIG. 15 meets criteria of area under receiver operating characteristic curve>0.90 and p value<0.05. Positive (red) points represent patients where seizures persisted post surgery. Negative (blue) points represent patients who were rendered seizure-free by ATL/AH. rs7900633 is a single nucleotide polymorphism (SNP) in which guanine is substituted for thymine on chromosome 10p.13. Heretofore, the clinical significance of rs7900633 has been unknown. In the current study, the prognostic value of down-regulation of temporal cortical rs7900633 expression for seizure-free outcome following ATL/AH is the first reported association between this SNP and epilepsy.

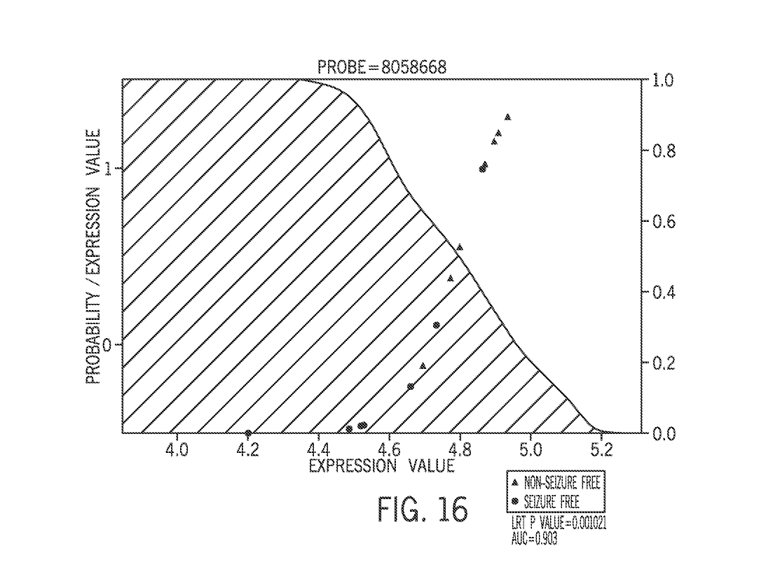

Targeted gene expression analysis has also detected significant down-regulation of rs8058668 temporal cortical expression associated with seizure-free outcome following ATL/AH. FIG. 16 illustrates a conditional probability plot for RNA Probe rs8058668, wherein FIG. 16 meets criteria of area under receiver operating characteristic curve>0.90 and p value<0.05. Positive (red) points represent patients where seizures persisted post surgery. Negative (blue) points represent patients who were rendered seizure-free by ATL/AH. rs8058668 is a single nucleotide polymorphism (SNP) located on chromosome 16q.24. Heretofore, the clinical significance of rs8058668 has been unknown. The prognostic value of relative down-regulation of rs8058668 temporal cortical expression for seizure-free outcome following ATL/AH is the first reported association between this SNP and epilepsy.

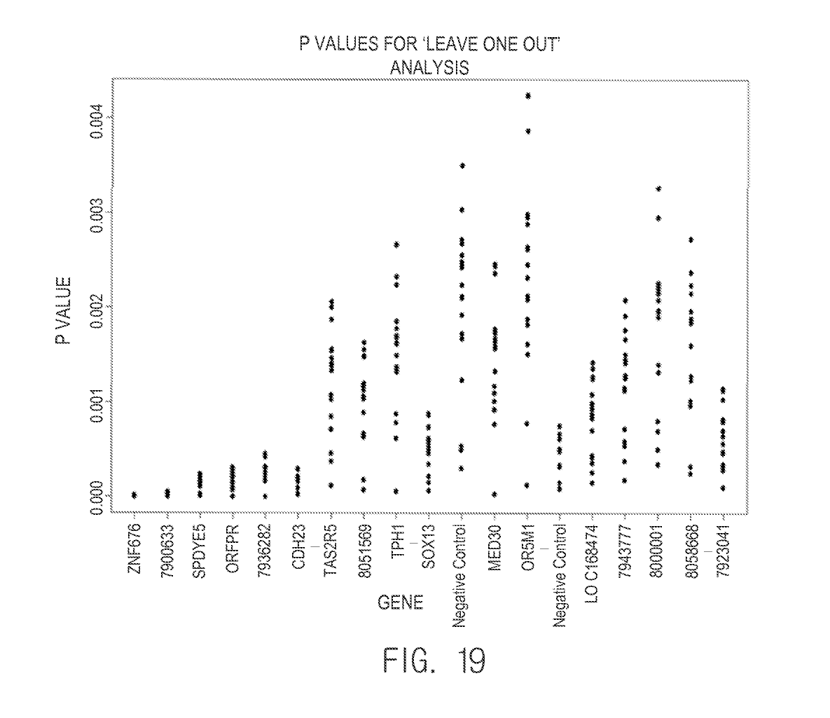

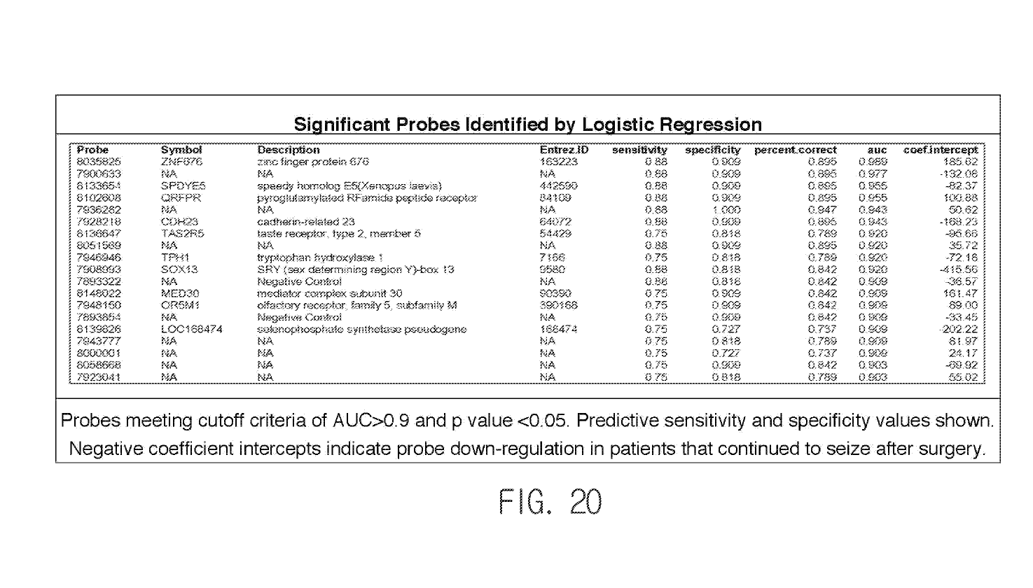

As illustrated in FIGS. 1-17 (Statistical event plots of gene & RNA probes), FIG. 19 (p values for ‘Leave Out’ Analysis) and FIG. 20 (Significant Probes by Logistic Regression), Applicants have found that downregulation or upregulation of ten (10) specific genes and seven (7) specific single nucleotide polymorphisms (SNPs) in the brain tissue of patients with medically intractable temporal lobe epilepsy provides an accurate predictor of which patients would be rendered seizure-free following ATL/AH. Referring now to FIG. 18 as an example with six of the aforementioned genes, downregulation of gene expression for one or more of the genes illustrated in red, namely CDH23, TPH1, SPDYE5, and/or TAS2R5, provides an accurate predictor of which patients would be rendered seizure-free following ATL/AH. Upregulation of gene expression for one or more of the genes illustrated in blue, namely MED30 and/or QRFPR, provides an accurate predictor of which patients would be rendered seizure-free following ATL/AH.

The genes and biological process pathways in human brain tissue and blood discussed hereinabove can further be used to broaden the scope “neurosurgical genomics” to include new biomarkers predictive of seizure outcome following ATL/AH. Furthermore, brain and/or whole blood gene expression (i.e. up-and/or down-regulation) which results from temporal lobectomy with amygdalohippocampectomy may be genetically engineered to obtain the clinically desired effect of seizure freedom by genomic expression obviating the need for neurosurgical operation. Applicants' method utilizing genetic engineering to produce a seizure-free outcome in temporal lobe epilepsy comprises anew field of “genomic neurosurgery”.

In summary, this invention documents the prognostic value for the cerebral cortical expression of 6 biological process pathways, 10 genes and 7 single nucleotide polymorphisms (SNP) for seizure outcome following anterior temporal lobectomy with amygdalohippocampectomy (ATL/AH). Modifications and adaptations of this invention as applied to human brain tissue and whole blood could establish new biological process pathway, gene and single nucleotide polymorphism biomarkers predictive of seizure outcome following ATL/AH.

While the preferred embodiments of the present invention have been illustrated in detail, it should be apparent that modifications and adaptations to those embodiments may occur to one skilled in the art without departing from the scope of the present invention as set forth herein.

1. A method to predict whether a patient afflicted with medically intractable temporal lobe epilepsy is likely to be rendered seizure-free following anterior temporal lobectomy with amygdalohippocampectomy (ATL/AH), comprising:
resecting lateral temporal cortex tissue from said patient;
isolating polynucleic acid samples from said tissue;
selecting a polynucleic acid;
producing a labeled target using a selected polynucleic acid probe;
determining an actual expression level of said selected polynucleic acid; and
wherein the relative expression of said selected polynucleic acid has predictive value with regard to seizure-free surgical outcome.

2. The method of claim 1, wherein said selected polynucleic acid comprises a gene.

3. The method of claim 2, wherein:
said selected polynucleic acid comprises gene Cadherin-related 23; and
down-regulation of cadherin-related 23 (CDH23) temporal cortical gene expression is associated with seizure-free outcome following ATL/AH.

4. The method of claim 2, wherein:
said selected polynucleic acid comprises gene tryptophan hydroxylase 1; and
down-regulation of tryptophan hydroxylase 1 (TPH1) temporal cortical gene expression is associated with seizure-free outcome following ATL/AH.

5. The method of claim 2, wherein:
said selected polynucleic acid comprises gene gene speedy homolog E5; and
down-regulation of speedy homolog E5 (SPDYE5) temporal cortical gene expression is associated with seizure-free outcome following ATL/AH.

6. The method of claim 2, wherein:
said selected polynucleic acid comprises gene taste receptor, type 2, member 5; and
down-regulation of the taste receptor, type 2, member 5 (TAS2R5) temporal cortical gene expression is associated with seizure-free outcome following ATL/AH.

7. The method of claim 2, wherein:
said selected polynucleic acid comprises gene mediator complex subunit 30; and
up-regulation of mediator complex subunit 30 (MED30) temporal cortical gene expression is associated with seizure-free outcome following ATL/AH.

8. The method of claim 2, wherein:
said selected polynucleic acid comprises gene pyroglutamylated RF amide peptide receptor; and
up-regulation of pyroglutamylated RF amide peptide receptor (QRFPR) temporal cortical gene expression was associated with seizure-free outcome following ATL/AH.

9. The method of claim 2, wherein:
said selected polynucleic acid comprises gene zinc finger protein 676; and
up-regulation of ZNF676 zinc finger protein 676 temporal cortical gene expression was associated with seizure-free outcome following ATL/AH.

10. The method of claim 2, wherein:
said selected polynucleic acid comprises gene sex determining region Y-box 13; and
down-regulation of SOX13 SRY (sex determining region Y)-box 13 temporal cortical gene expression was associated with seizure-free outcome following ATL/AH.

11. The method of claim 2, wherein:
said selected polynucleic acid comprises gene olfactory receptor, family 5, subfamily M, member 1; and
up-regulation of OR5M1 olfactory receptor, family 5, subfamily M, member 1 temporal cortical gene expression was associated with seizure-free outcome following ATL/AH.

12. The method of claim 2, wherein:
said selected polynucleic acid comprises gene Selenophosphate Synthetase 1 Pseudogene 1; and
relative down-regulation of Selenophosphate Synthetase 1 Pseudogene 1 (SEPHS1P) temporal cortical gene expression is associated with seizure-free outcome following ATL/AH.

13. The method of claim 1, wherein said selected polynucleic acid comprises a single nucleotide polymorphism.

14. The method of claim 13, wherein:
said selected polynucleic acid comprises RNA Probe rs8051569; and
up-regulation of RNA Probe rs8051569 is associated with seizure-free outcome following ATL/AH.

15. The method of claim 13, wherein:
said selected polynucleic acid comprises RNA Probe rs7923041; and
up-regulation of RNA Probe rs7923041 is associated with seizure-free outcome following ATL/AH.

16. The method of claim 13, wherein:
said selected polynucleic acid comprises RNA probe, rs7943777; and
up-regulation of RNA probe, rs7943777 is associated with seizure-free outcome following ATL/AH.

17. The method of claim 13, wherein:
said selected polynucleic acid comprises RNA probe rs8000001; and
up-regulation of RNA probe rs8000001 is associated with seizure-free outcome following ATL/AH.

18. The method of claim 13, wherein:
said selected polynucleic acid comprises RNA probe rs7936282; and
down-regulation of RNA probe rs7936282 is associated with seizure-free outcome following ATL/AH.

19. The method of claim 13, wherein:
said selected polynucleic acid comprises RNA probe rs7900633; and
down-regulation of temporal cortical RNA probe rs7900633 expression is associated with seizure-free outcome following ATL/AH.

20. The method of claim 13, wherein:
said selected polynucleic acid comprises RNA Probe rs8058668; and down-regulation of RNA probe rs8058668 temporal cortical expression is associated with seizure-free outcome following ATL/AH.
